(19) United States
(12) Patent Application Publication
(10) Pub. No.: US 2025/0215465 A1
(43) Pub. Date: Jul. 3, 2025

(54) ENANTIOSELECTIVE METHODS FOR PREPARING CHIRAL AMINE INTERMEDIATES

(71) Applicant: UNIVERSITY OF THE WITWATERSRAND, JOHANNESBURG, Johannesburg (ZA)

(72) Inventors: Daniel Petzer PIENAAR, Johannesburg (ZA); Dean BRADY, Johannesburg (ZA); Josephine Tshegofatso MABOYA, Johannesburg (ZA)

(21) Appl. No.: 18/850,713

(22) PCT Filed: Apr. 4, 2023
(86) PCT No.: PCT/ZA2023/050020

§ 371 Date: Sep. 25, 2024

(30) Foreign Application Priority Data
Apr. 4, 2022 (ZA) 2022/03797

(51) Int. Cl. C12P 13/00; C07C 227/20; C12N 9/10; C12P 41/00

(52) U.S. Cl. CPC C12P 13/001; C07C 227/20; C12N 9/1096; C12P 41/007

(57) ABSTRACT

A method for preparing chiral amines useful as pharmaceutical intermediates is provided. The method comprises contacting an ester of formula I in which R₁ is a methyl or an ethyl group; R₂ is a linear or branched C₁-C₄ alkyl group; and n is 0 or 1 with an enantioselective ω-transaminase in the presence of an amino donor so that the ω-transaminase catalyses the enantioselective transfer of an amino group from the amino donor to the α- or β-ketone group of the ester of formula I to produce an amino ester product with enantiomeric excess of a selected enantiomer.

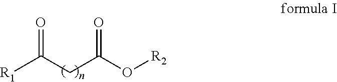

### CROSS-REFERENCE(S) TO RELATED APPLICATIONS

This application claims priority from South African provisional patent application number 2022/03797 filed on 4 Apr. 2022, which is incorporated by reference herein.

### FIELD OF THE INVENTION

This invention relates to enantioselective methods for preparing chiral amine compounds. In particular, it relates to biocatalytic methods for producing chiral amine intermediates and the active ingredients made therefrom.

### BACKGROUND TO THE INVENTION

Many intermediate compounds in the synthesis of pharmaceutical active ingredients, agrichemicals, food and feed additives and polymers are being produced as racemic mixtures of enantiomers. At best, just 50% of the material, i.e., the desired enantiomer, can then be further used. Discarding the undesired enantiomer in cases where it cannot be recycled or further racemized generates a lot of waste. This is the case in the production of the active pharmaceutical ingredient (API) ethambutol, for example.

Ethambutol 1 is listed as an essential World Health Organization (WHO) drug for use in combination drug therapy against tuberculosis (TB). Ethambutol has the following chemical structure:

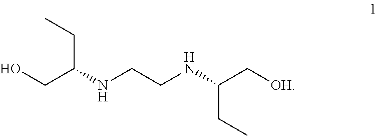

Ethambutol is a mainstay of TB treatment as it plays a crucial role in lowering the incidence of the development of mycobacterial resistance against other treatments, such as isoniazid, rifampin and pyrazinamide, which are used in combination with ethambutol in standard TB treatment. Ethambutol acts by inhibiting normal mycobacterial cell wall assembly, by targeting the arabinosyl transferase enzyme responsible for arabinogalactan biosynthesis, an essential cell wall component. The active compound has two key (S)-amino-alcohol units. The stereochemistry of these units (S, S) is essential for ethambutol's efficacy, with the (R, R) mirror image stereoisomer being about 500 times less active against TB proliferation.

Known processes for synthesizing ethambutol include a critical chiral resolution step of racemic amino alcohol, by first converting the intermediate racemic (50/50 R and S) mixture of the amino alcohol into the corresponding (+)-tartrate salts (Scheme 1), as described in United States (US) U.S. Pat. No. 3,651,144. The required(S) salt is selectively precipitated and only this component can be subsequently used. This process creates waste, as more than 50% of the racemic compound (the (R) component) is effectively discarded as it cannot be economically recycled or racemized and reused. It also results in a low yield. A maximum yield of 31% of the desired (S)-enantiomer 3 can be obtained from the racemic mixture after completion of laborious recrystallization and subsequent decomplexation processes.

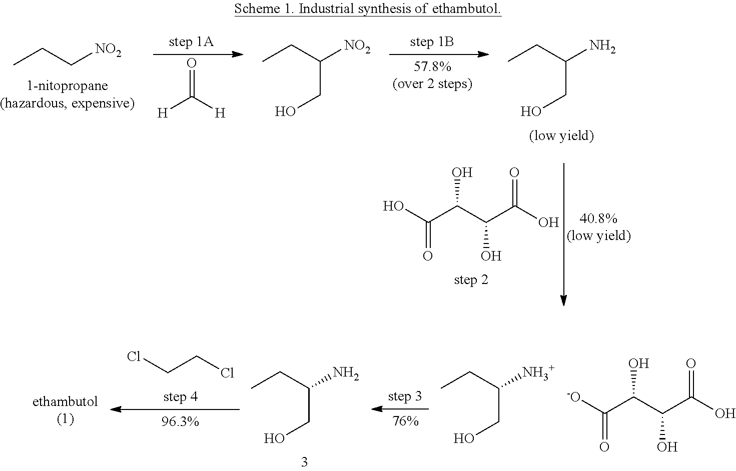

Ethambutol can be prepared stereoselectively from an ester of the unnatural amino acid (S)-2-aminobutanoic acid 2 by harsh reduction to obtain amino alcohol 3 followed by reaction with 1,2-dichloroethane to produce ethambutol 1 (Scheme 2), as described in U.S. Pat. No. 3,979,457.

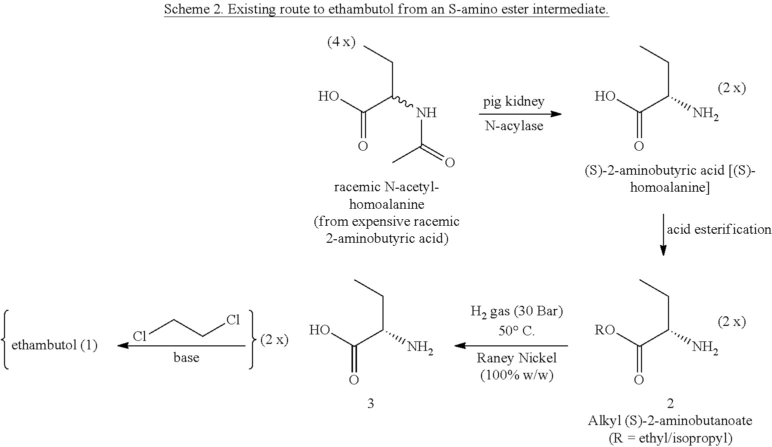

The synthesis of ethambutol shown in Scheme 2 starts from an unnatural amino acid (racemic 2-aminobutyric acid) which is relatively expensive. A theoretical maximum yield of 50% of the desired (S) amino ester enantiomer 2 can be obtained using classic bio-resolution with an N-acylase enzyme as per Scheme 2. Furthermore, ethambutol production using an amino acid as a starting material and an intermediate requires costly, large-scale ion exchange chromatography to purify the respective amino acids. This is made more cumbersome and expensive by the need to dry the amino acid product to remove water prior to further use.

Russian patent publication number RU27212231 (C1) describes an alternative method for producing ethambutol from racemic 2-aminobutan-1-ol. The method includes protecting the amino group by reacting it with carboxybenzyl chloride in the presence of sodium hydroxide to obtain the N-carbobenzoxy derivative of 2-aminobutan-1-ol. The alcohol group of the derivative is then stereoselectively acylated with ethyl acetate and this reaction is catalysed by lipase PPL. The (S)-enantiomer is recovered by reduction to obtain (S)-2-aminobutan-1-ol, which is alkylated with 1,2-dichloroethane to form the product. The starting material for ethambutol production according to this method is nitropropane. The lipase resolution step of the method produces a maximum 50% theoretical yield of the desired (S)-enantiomer intermediate. The protective N-carbobenzoxy group is relatively expensive to install. Also, a palladium-catalysed hydrogenation step is required to deprotect the N-carbobenzoxy group, to get to the desired free amino alcohol compound.

Another API made from an unnatural amino acid is dolutegravir. Dolutegravir (5 in Scheme 3 below) is a potent, new generation integrase strand transfer inhibitor drug against human immunodeficiency virus (HIV) infection. It has been approved by the FDA for the treatment of children born with HIV, from as young as 4 weeks old. Dolutegravir is recommended for first line antiretroviral treatment (ART) for treatment-naïve individuals. It is also recommended that people experiencing side-effects with other treatment regimens switch to the new regimen of dolutegravir, tenofovir disoproxil fumarate and lamivudine, known as TLD. This is a fixed-dose combination including one integrase inhibitor (dolutegravir) and two nucleoside/nucleotide reverse transcriptase inhibitors (NRTIs). The new regimen that includes dolutegravir has several advantages, including the fact that it has fewer interactions with other medicines, such as those used to treat tuberculosis, which is a common co-morbidity.

The current industrial synthesis of dolutegravir that is considered most efficient to date is depicted in Scheme 3 and described in European patent publication number EP 2 320 908 B9.

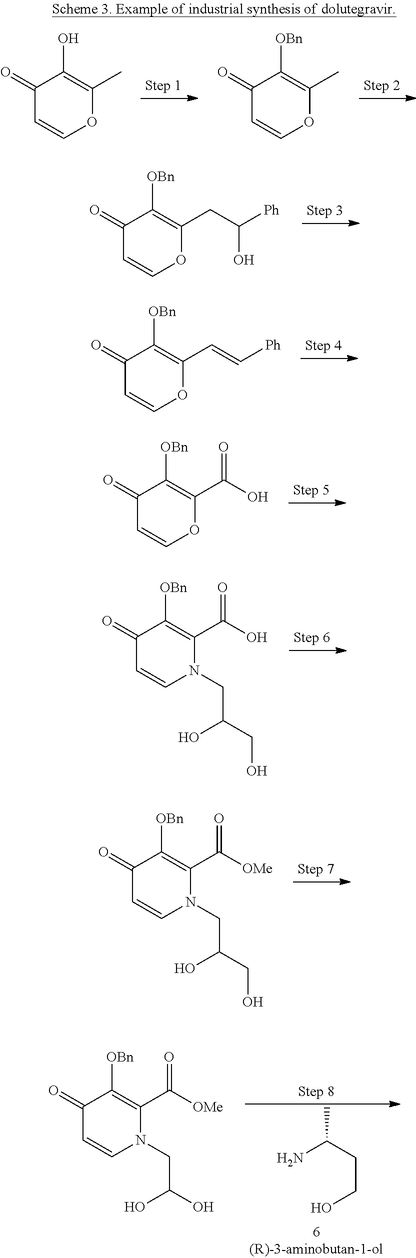

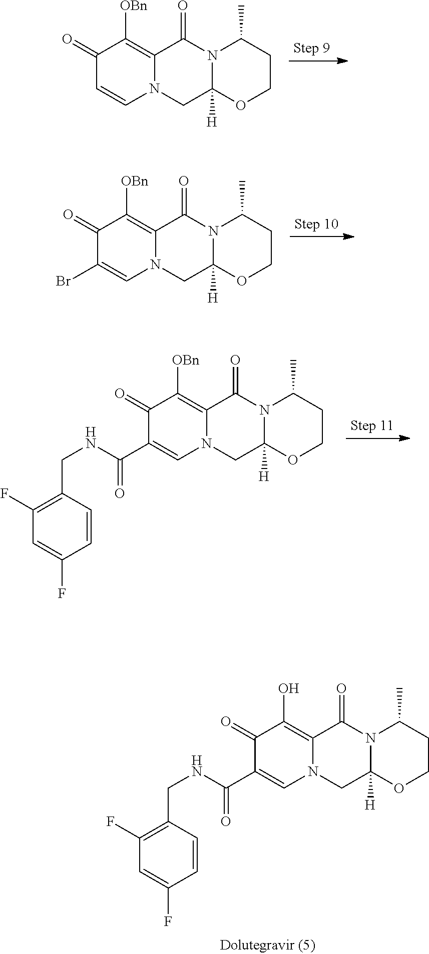 [SMILES: C[C@@H]1CCO[C@H]2Cn3ccc(=O)c(OCc4ccccc4)c3C(=O)N12 ; C[C@@H]1CCO[C@H]2Cn3cc(Br)c(=O)c(OCc4ccccc4)c3C(=O)N12 ; C[C@@H]1CCO[C@H]2Cn3cc(C(=O)NCc4ccc(F)cc4F)c(=O)c(OCc4ccccc4)c3C(=O)N12 ; C[C@@H]1CCO[C@H]2Cn3cc(C(=O)NCc4ccc(F)cc4F)c(=O)c(O)c3C(=O)N12]

The synthesis depends on access to a critical intermediate, (R)-3-amino-1-butanol (6) (step 8 in Scheme 3). The amino alcohol (6) cannot be readily prepared from naturally occurring amino acids. For every kilogram of dolutegravir produced, 463 g of (R)-3-aminobutan-1-ol (6) is required. Accordingly, a less costly process for its production will reduce the overall cost of producing dolutegravir.

Generally, there is a need for environmentally friendly or “green” and less expensive alternatives to conventional chemical processes to produce specific chiral amines as intermediates or precursors and the bioactive compounds made therefrom.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with an aspect of the invention there is provided a method for preparing chiral amines, comprising contacting an ester of formula I:

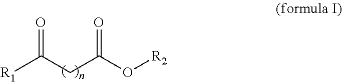

in which R₁ is a methyl or an ethyl group;
R₂ is a linear or branched C₁-C₄ alkyl group; and
n is 0 or 1
   with an enantioselective ω-transaminase in the presence of an amino donor so that the ω-transaminase catalyses the enantioselective transfer of an amino group from the amino donor to an α-ketone group when n is 0 or an β-ketone group when n is 1 of the ester of formula I to produce an amino ester product with enantiomeric excess of a selected enantiomer.

The enantiomeric excess may be at least 70%, preferably at least 95% and more preferably at least 99%. The amino ester product may be substantially enantiomerically pure. The selected enantiomer may be recovered with a yield of at least 80 mol %.

The enantioselective ω-transaminase may be an (S)-selective ω-transaminase for a selected ester of formula I to produce enantiomeric excess of an (S)-amino ester enantiomer. Alternatively, the enantioselective ω-transaminase may be an (R)-selective ω-transaminase for a selected ester of formula I to produce enantiomeric excess of an (R)-amino ester enantiomer.

The amino donor may be isopropyl amine. The contact step may be carried out in a buffer solution. The buffer solution may include pyridoxal phosphate (PLP) as co-factor. The pH of the buffer solution may range between about 7.1 and 7.5. The contact step may be carried out at a temperature within the range of about 20° C. to 40° C., and preferably at about 30° C.

The method may include reducing the selected amino ester enantiomer to the corresponding amino acid or amino alcohol in a manner which substantially avoids racemization occurring.

Hydrolysis of the selected amino ester enantiomer to the corresponding amino acid may be carried out with a catalytic amount of a base in a solvent. The solvent may be water or an alcohol-based solvent. Hydrolysis of the selected enantiomer to the corresponding amino acid may be carried out at room temperature. Alternatively, the hydrolysis of the selected amino ester enantiomer to the corresponding amino acid may be enzyme-catalysed by a lipase or protease enzyme, for example.

The reduction of the selected amino ester enantiomer to the corresponding amino alcohol may be carried out using hydrogen gas over a Raney Nickel catalyst. The reduction of the selected amino ester enantiomer to the corresponding amino alcohol may be carried out in a solvent with a reducing agent. The reducing agent may be a borohydride reagent such as sodium borohydride with a suitable Lewis acid catalyst such as boron trifluoride, or calcium borohydride. Alternatively, when using sodium borohydride, iodine may be added as a catalyst. The solvent is preferably an ethereal solvent. The reduction of the selected enantiomer to the corresponding amino alcohol may be carried out at a temperature ranging from room temperature to about 70° C.

R₂ may be a methyl, ethyl, isopropyl, n-butyl, sec-butyl, isobutyl, or tert-butyl group.

When n is 0, R₁ may be an ethyl group so that the ester of formula I is a C₁-C₄ alkyl 2-oxobutyrate. The ω-transaminase may be an (S)-selective ω-transaminase configured to catalyse the production of an amino ester product with enantiomeric excess of C₁-C₄ alkyl (S)-2-aminobutanoate. R₂ may be an ethyl or an isopropyl group. The (S)-selective ω-transaminase for ethyl or isopropyl 2-oxobutyrate may be the (S)-selective amine transaminases (ATA) enzymes Prozomix ATA 230 or Prozomix ATA 254. The enantiomeric excess of the ethyl or isopropyl (S)-2-aminobutanoate may be at least 99%. The method may include hydrolysing the ethyl or isopropyl (S)-2-aminobutanoate to (S)-2-aminobutyric acid ((S)-homoalanine), which is useful for the synthesis of levetiracetam or brivaracetam. The method may include converting (S)-2-aminobutyric acid to levetiracetam or brivaracetam. Alternatively, the method may include reducing the ethyl or isopropyl (S)-2-aminobutanoate to (S)-2-aminobutan-1-ol, which is useful for the synthesis of ethambutol. The method may include converting (S)-2-aminobutan-1-ol to ethambutol.

Alternatively, when n is 0 and R₁ is an ethyl group, the ω-transaminase may be an (R)-selective ω-transaminase configured to catalyse the production of an amino ester product with enantiomeric excess of C₁-C₄ alkyl (R)-2-aminobutanoate. R₂ may be an ethyl or an isopropyl group. The (R)-selective ω-transaminase for ethyl or isopropyl 2-oxobutyrate may be the (R)-selective ATA enzyme Prozomix ATA 239 or a wild-type (R)-selective transaminase. The wild-type (R)-selective transaminase may be isolated from one or more of Neosartorya fischeri, Aspergillus fumigatus or Aspergillus terreus. The enantiomeric excess of the ethyl or isopropyl (R)-2-aminobutanoate may be at least 70%. The method may include hydrolysing the ethyl or isopropyl (R)-2-aminobutanoate to (R)-2-aminobutyric acid ((R)-homoalanine). Alternatively, the method may include reducing the ethyl or isopropyl (R)-2-aminobutanoate to (R)-2-aminobutan-1-ol.

The method may include preparing the ester of formula I in which n is 0 and R₁ is an ethyl group by reacting diethyl or diisopropyl oxalate with ethyl magnesium bromide to produce an ethyl or isopropyl 2-oxobutyrate.

When n is 1, R₁ may be a methyl group so that the ester of formula I is an C₁-C₄ alkyl (3-oxobutyrate. The ω-transaminase may be an (R)-selective ω-transaminase configured to catalyse the production of an amino ester product with enantiomeric excess of C₁-C₄ alkyl ((R)-3-aminobutanoate. R₂ may be an ethyl or isopropyl group. The (R)-selective ω-transaminase for ethyl or isopropyl 3-oxobutyrate may be an (R)-selective ATA enzyme selected from one or more of Prozomix ATA 234, Prozomix ATA 241, Prozomix ATA 254 or Prozomix ATA 261 or an (R)-selective wild type enzyme isolated from Neosartorya fischeri, Aspergillus fumigatus or Aspergillus terreus. The method may include hydrolysing the ethyl or isopropyl (R)-3-aminobutanoate to (R)-3-aminobutyric acid. Alternatively, the method may include reducing the ethyl or isopropyl (R)-3-aminobutanoate to (R)-3-aminobutan-1-ol, which is useful for the synthesis of dolutegravir. The method may include converting aminobutan-1-ol to dolutegravir

Alternatively, when n is 1 and R₁ is a methyl group, the ω-transaminase may be an (S)-selective ω-transaminase configured to catalyse the production of an amino ester product with enantiomeric excess of C₁-C₄ alkyl (S)-3-aminobutanoate. The method may include a hydrolysing the C₁-C₄ alkyl (S)-3-aminobutanoate to (S)-3-aminobutyric acid. Alternatively, the method may include reducing the C₁-C₄ alkyl (S)-3-aminobutanoate to (S)-3-aminobutan-1-ol.

In accordance with a second aspect of the invention there is provided a method of preparing levetiracetam or brivaracetam, the method comprising the steps of:
contacting C₁-C₄ alkyl 2-oxobutyrate with an (S)-selective ω-transaminase in the presence of an amino donor to produce substantially enantiomerically pure C₁-C₄ alkyl (S)-2-aminobutanoate;
hydrolysing the C₁-C₄ alkyl (S)-2-aminobutanoate to (S)-2-aminobutyric acid; and converting (S)-2-aminobutyric acid to levetiracetam or brivaracetam.

In accordance with a third aspect of the invention there is provided a method of preparing ethambutol, the method comprising the steps of:
contacting C₁-C₄ alkyl 2-oxobutyrate with an (S)-selective ω-transaminase in the presence of an amino donor to produce substantially enantiomerically pure C₁-C₄ alkyl (S)-2-aminobutanoate;
reducing the C₁-C₄ alkyl (S)-2-aminobutanoate to (S)-2-aminobutan-1-ol; and
converting the (S)-2-aminobutan-1-ol to ethambutol.

In accordance with a fourth aspect of the invention there is provided a method of preparing dolutegravir, the method comprising the steps of
contacting C₁-C₄ alkyl 3-oxobutyrate with an (R)-selective ω-transaminase in the presence of an amino donor to produce substantially enantiomerically pure C₁-C₄ alkyl (R)-3-aminobutanoate;
reducing the C₁-C₄ alkyl (R)-3-aminobutanoate to (R)-3-aminobutan-1-ol; and
converting the (R)-3-aminobutan-1-ol to dolutegravir.

Embodiments of the invention will now be described, by way of example only, with reference to the accompanying drawings.

### BRIEF DESCRIPTION OF THE DRAWINGS

In the drawings:

FIG. 1 is a bar graph of the area under the elution peaks for the isopropyl 2-aminobutyrate product obtained with different Prozomix ATA enzymes and analysed with achiral High Performance Liquid Chromatography (HPLC);

FIG. 2 is a plot of the results of Ultra-high Performance Liquid Chromatography-MS (UPLC-MS) analysis of chemically synthesised racemic isopropyl 2- aminobutyrate;

FIG. 3 is a plot of the results of UPLC-MS analysis of chemically synthesised (S)-isopropyl 2-aminobutyrate

FIG. 4 is a plot of the results of UPLC-MS analysis of the isopropyl 2-aminobutyrate product from Prozomix ATA 230;

FIG. 5 is a plot of the results of UPLC-MS analysis of the isopropyl 2-aminobutyrate product from Prozomix ATA 254;

FIG. 6 is a plot of the results of UPLC-MS analysis of the isopropyl 2-aminobutyrate product from Prozomix ATA 239;

FIG. 7 is a plot of the results of LC-MS analysis of the isopropyl 3-aminobutyrate product from Prozomix ATA 235;

FIG. 8 is a bar graph of the area under the elution peaks for the isopropyl 3-aminobutyrate product obtained with different ATA enzymes and analysed with Liquid Chromatography-Mass Spectrometry (LC-MS);

FIG. 9 is a UPLC-UV (254 nm) chromatogram for racemic, derivatised isopropyl 3-aminobutyrate;

FIG. 10 is a UPLC-UV (254 nm) chromatogram for derivatised isopropyl(S)-3-aminobutyrate;

FIG. 11 is a chromatogram of the UPLC-UV (254 nm) analysis of product from Prozomix ATA 254;

FIG. 12 is a chromatogram of the UPLC-UV (254 nm) analysis of product from Prozomix ATA 261;

FIG. 13 is a chromatogram of the UPLC-UV (254 nm) analysis of product from Prozomix ATA 234;

FIG. 14 is a chromatogram of the UPLC-UV (254 nm) analysis of product from Prozomix ATA 241.

FIG. 15 is a proton NMR spectrum of chemically synthesised 4-nitrobenzamide derivative of the (S)-2-amino isopropyl ester; and

FIG. 16 is a proton NMR spectrum of the 4-nitrobenzamide derivative of the Prozomix ATA 254 enzyme produced (S)-2-amino isopropyl ester.

### DETAILED DESCRIPTION WITH REFERENCE TO THE DRAWINGS

Methods for preparing compounds including a chiral amine functionality and methods for preparing the bioactive compounds derived from such chiral amines are provided. More specifically, the methods are for preparing chiral amino ester compounds that are useful for producing several different bioactive molecules. The methods involve enzyme-catalysed and enantioselective biotransformation of α- or β-keto esters to α- or β-amino esters. The α- or β-keto esters are contacted with either an (R)- or (S)-selective ω-transaminase for the specific α- or β-keto ester in the presence of an amino donor to convert the α- or β-keto esters to either the (R)- or (S)-enantiomer of an α- or β-amino ester.

Amine transaminases (ATAs) are enzymes that effectively catalyse transamination reactions between amino acids and keto acids under biological conditions in cells. It has now been surprisingly found that (R)- or (S)-selective ω (omega)-transaminases, which may be native or wild-type enzymes or variants thereof, can be successfully employed to produce highly enantiomerically enriched amino ester compounds from keto ester compounds as substrates. Therefore, it was surprisingly found that the ω-transaminases do not just act on keto acids. Advantageously, the amino ester enantiomers produced in this manner can be recovered in higher yields in comparison to amino acid enantiomers.

ω-Transaminases are a group of pyridoxal-5′-phosphate (PLP) dependant enzymes capable of transferring an amino group from an amino donor molecule to an amino acceptor carbonyl group. This transamination reaction is achieved through two half-reactions, where the amino group is first transferred into the PLP initially bound to the enzyme to form pyridoxamine-5′-phosphate (PMP), which then reacts with the amino acceptor to form the final product and recover the coenzyme in its initial state, bound to the protein. ω-Transaminases can be used in vitro on unnatural substrates in stereoselective synthesis to obtain substantially enantiopure amines from the corresponding prochiral ketone. The reaction can provide a theoretical yield of 100% if the equilibrium is shifted in favour of the transamination reaction. This can be done with either removal of the co-product (for example, acetone) or a large excess of the amino donor (for example, isopropylamine).

ω-Transaminases can be used in a biocatalytic and enantioselective method for preparing chiral amine compounds which may be pharmaceutical intermediates. The unnatural substrate of the ω-transaminase catalysed reaction is a keto ester, more specifically an α- or β-ketone ester. The method comprises contacting an ester of formula I:

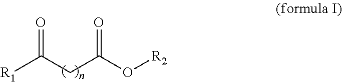

in which R₁ is a methyl or an ethyl group;
R₂ linear or branched C₁-C₄ alkyl group; and
n is 0 or 1
   with an enantioselective ω-transaminase in the presence of an amino donor, the enantioselective ω-transaminase being configured to catalyse the enantioselective transfer of an amino group from the amino donor to an α- or β-ketone group of the ester of formula I to produce an amino ester product with an enantiomeric excess of a selected enantiomer. Naturally, when n is 0, the amino group is transferred to the α-ketone group of the ester of formula I and when n is 1, the amino group is transferred to the β-ketone group of the ester of formula I. The enantiomeric excess may vary depending on the type of enantioselective ω-transaminase used, the reaction conditions and the types and concentration of the reagents in the contact step. The enantiomeric excess may be at least about 70%, preferably at least about 95% and more preferably at least about 99%. Reaction conditions and reagent concentrations may be optimised so that the amino ester product is substantially enantiomerically pure.

The preferred substrate for the enantioselective ω-transaminases are four-carbon or butyl esters, i.e., n is 1 when R₁ is a methyl group, and n is 0 when R₁ is an ethyl group. Therefore, the substrate may be a C₁-C₄ alkyl 2-oxobutyrate or a C₁-C₄ alkyl 3-oxobutyrate. More specifically, R₂ may be a methyl, ethyl, isopropyl, n-butyl, sec-butyl, isobutyl or tert-butyl group and preferably an ethyl or isopropyl group.

The enantioselective ω-transaminase may be an (S)-selective ω-transaminase configured to produce enantiomeric excess of an (S)-amino ester enantiomer from the selected keto ester substrate. Enzyme screening revealed that (S)-selective ATA enzymes Prozomix ATA 230 or Prozomix ATA 254 supplied by Prozomix Limited produce (S)-amino ester enantiomers from α-keto ester substrates in exceptionally high enantiomeric excess. Alternatively, the enantioselective ω-transaminase may be an (R)-selective ω-transaminase to produce enantiomeric excess of an (R)-amino ester enantiomer. For example, the (R)-selective ATA enzyme ATA 239 supplied by Prozomix Limited, or a wild-type (R)-selective transaminase isolated from one or more of Neosartorya fischeri, Aspergillus fumigatus or Aspergillus terreus may be used to obtain considerable enantiomeric excess with α-keto ester substrates. With β-keto ester substrates, the ATA enzymes Prozomix ATA 234, Prozomix ATA 241, Prozomix ATA 254 or Prozomix ATA 261 supplied by Prozomix Limited produced exceptionally high enantiomeric excess of the corresponding (R)-amino ester enantiomer.

The amino donor may be isopropyl amine and it may be present in excess to displace the equilibrium of the reaction to favour the production of the desired enantiomer. The ratio of the ester of formula I as the substrate to the amino donor may be in the order of about 0.02:1 (i.e., 1:50), for example. The contact step may be carried out in a suitable buffer solution with a pH ranging between about 7.1 and 7.5. The buffer may comprise 100 mM potassium dihydrogen phosphate and the pH may be adjusted with concentrated NaOH. The buffer further may include 0.5 g/L of pyridoxal phosphate (PLP) as co-factor. The contact step may be carried out at a modest temperature, preferably within the range of about 20° C. to 40° C., more preferably at 30° C. The contact step may be carried out for about 1-48 hours, preferably for about 1-24 hours. The contact step reaction time varies depending on the temperature and the enzymes employed.

The desired (R) or (S) amino ester enantiomer may be recovered with a yield of at least about 80 mol %. The recovery step may include stopping the reaction by adding brine, sodium bicarbonate and a suitable organic solvent for the product to the reaction mixture. The resultant mixture is mixed and then centrifuged to recover the product from the top organic layer. The solvent used in the recovery may be acetonitrile, for example.

Following recovery, the desired (R) or (S) amino ester enantiomer may be hydrolysed to the corresponding (R)- or (S)-amino acid or reduced to the corresponding (R)- or (S)-amino alcohol without substantial racemization occurring. The respective hydrolysis or reduction reactions and reagents may be selected to ensure that racemization does not occur.

The amino ester enantiomer may be hydrolysed to the corresponding amino acid enantiomer by way of base-catalysed or hydroxide-catalysed hydrolysis in water or an alcohol solvent at room temperature. A strong base such as potassium hydroxide in methanol can, for example, be used. Alternatively, the hydrolysis may be enzyme-catalysed, and any suitable lipase or protease enzyme may be used.

Alternatively, the amino ester enantiomer may be reduced to the corresponding amino alcohol enantiomer with hydrogen gas over a Raney Nickel catalyst or in a solvent with a suitable reducing agent. The hydrogen gas pressure may be in the range of 1 to 100 bar. The reducing agent may be a borohydride reagent such as excess sodium borohydride or calcium borohydride in an ether solvent and with a suitable Lewis acid catalyst such as boron trifluoride (and with additives lithium chloride or calcium chloride, for example) or with an iodine catalyst. The reaction temperature may be selected from room temperature to a temperature of about 70° C.

Relatively mild, less complex and more cost-effective synthetic routes toward pharmaceutical intermediates of essential anti-TB medicine, ethambutol, and antiepileptic drugs, levetiracetam and brivaracetam are provided which involve the above described stereoselective biocatalytic step, particularly with an (S)-selective ω-transaminase. The substrate for the biocatalytic step may be a C₁-C₄ alkyl 2-oxobutyrate or a C₁-C₄ alkyl 3-oxobutyrate. When the substrate is an ester of formula I in which n is 0 and R₁ is an ethyl group (i.e., ethyl or isopropyl 2-oxobutyrate), the substrate itself may first be prepared via the Grignard reaction of the relatively inexpensive diethyl or diisopropyl oxalate with ethyl magnesium bromide at 0° C. to produce ethyl or isopropyl 2-oxobutyrate. The ethyl or isopropyl 2-oxobutyrate is then subjected to biotransformation with an (S) -selective ω-transaminase to produce an amino ester product with enantiomeric excess of ethyl or isopropyl (S)-2-aminobutanoate. Accordingly, ethyl or isopropyl (S)-2-aminobutanoate may be stereoselectively synthesised with an (S)-selective ω-transaminase in the presence of excess amino donor such as isopropyl amine and in a suitable buffer. It was found that an enantiomeric excess of at least 99% can be achieved with (S)-selective ATA enzymes Prozomix ATA 230 or Prozomix ATA 254 supplied by Prozomix Limited.

The ethyl or isopropyl (S)-2-aminobutanoate may then be reduced to (S)-2-aminobutyric acid ((S)-homoalanine), which can be further used to synthesise levetiracetam or brivaracetam. The facile base hydrolysis may be carried out in a methanolic solution of sodium hydroxide at room temperature.

Accordingly, a method of preparing levetiracetam or brivaracetam is provided which comprises the steps of contacting a C₁-C₄ alkyl 2-oxobutyrate with an (S)-selective ω-transaminase in the presence of an amino donor to produce substantially enantiomerically pure C₁-C₄ alkyl (S)-2-aminobutanoate, reducing the C₁-C₄ alkyl (S)-2-aminobutanoate to (S)-2-aminobutyric acid; and finally converting (S)-2-aminobutyric acid to levetiracetam or brivaracetam or using (S)-2-aminobutyric acid to prepare levetiracetam or brivaracetam. The C₁-C₄ alkyl 2-oxobutyrate may be ethyl of isopropyl 2-oxobutyrate. Ethyl or isopropyl 2-oxobutyrate may be prepared according to the Grignard reaction of diethyl or diisopropyl oxalate with ethyl magnesium bromide as described above.

When the (S)-amino ester, C₁-C₄ alkyl (S)-2-aminobutanoate, is instead reduced to the corresponding (S)-homoalaninol, (S)-2-aminobutan-1-ol, it can be further used to synthesise ethambutol. The simple reduction of the ester to the corresponding alcohol may be carried out with excess sodium borohydride in tetrahydrofuran with additives lithium chloride or calcium chloride and at a temperature ranging between room temperature and about 70° C.

Accordingly, the method of preparing ethambutol comprises the steps of contacting C₁-C₄ alkyl 2-oxobutyrate with an (S)-selective ω-transaminase in the presence of an amino donor to produce substantially enantiomerically pure C₁-C₄ alkyl (S)-2-aminobutanoate, reducing the C₁-C₄ alkyl (S) -2-aminobutanoate to (S)-2-aminobutan-1-ol, and converting the (S)-2-aminobutan-1-ol to ethambutol (i.e., using (S)-2-aminobutan-1-ol as an intermediate for preparing ethambutol). The C₁-C₄ alkyl 2-oxobutyrate may be ethyl of isopropyl 2-oxobutyrate. Again, the ethyl or isopropyl 2-oxobutyrate may be prepared according to the Grignard reaction of diethyl or diisopropyl oxalate with ethyl magnesium bromide.

Exemplary synthetic routes to ethambutol and levetiracetam and brivaracetam are shown in Scheme 4.

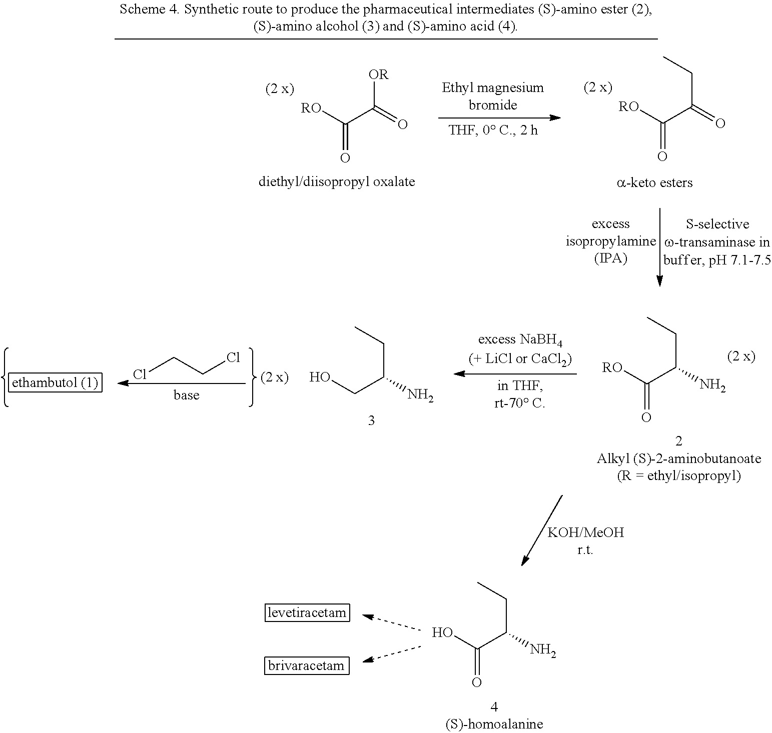

The same substrate, C₁-C₄ alkyl 2-oxobutyrate, can be used to produce the (R)-amino ester enantiomer, C₁-C₄ alkyl (R)-2-aminobutanoate, by incorporating an (R)-selective ω-transaminase catalysed step. When the substrate is ethyl or isopropyl 2-oxobutyrate, it was found that an enantiomeric excess of at least about 70% is achievable when this reaction is catalysed with (R)-selective ATA enzyme Prozomix ATA 239 supplied by Prozomix Limited, for example. Other well-known wild-type (R)-selective ATA's can be used, such as those isolated from Neosartorya fischeri, Aspergillus fumigatus or Aspergillus terreus. As with the (S)-amino ester product and under the same reaction conditions, the C₁-C₄ alkyl (R)-2-aminobutanoate may be reduced to (R)-2-aminobutyric acid ((R)-homoalanine) or to (R)-2-aminobutan-1-ol ((R)-homoalaninol). These (R)-amino acid and (R)-amino alcohol intermediates may also be used in synthetic routes towards other therapeutic or agrichemical compounds or in other applications.

The enantioselective α-homoalaninol and α-homoalanine production processes described herein are more cost-effective in comparison to existing methods. For example, the starting material, racemic homoalanine, used in the existing method of producing ethambutol shown in Scheme 3is much more expensive than diisopropyl or diethyl oxalate, which can be sustainably produced from the facile esterification of oxalic acid with inexpensive isopropanol. Also, an additional N-acylation step needs to be carried out to produce the N-acetyl starting material for the N-acylase enzyme in the process of Scheme 3. Most importantly, a much higher yield of the desired (S)-amino ester enantiomer (i.e., theoretically a 100% yield) can be obtained by the methods described herein, as opposed to a maximum yield of 50% via a classic biocatalytic resolution using an N-acylase enzyme. Accordingly, the methods described herein produces less waste in comparison to prior synthetic routes to ethambutol.

Useful (R) or (S) β-homoalaninol and β-homoalanine intermediates may also be produced when the substrate for the (R)- or (S)-selective ω-transaminase is C₁-C₄ alkyl 3-oxobutyrate. Accordingly, the ester of formula I in which n is 1 and R₁ is a methyl group (i.e. C₁-C₄ alkyl 3-oxobutyrate) may be contacted with an (R)-selective ω-transaminase to produce an amino ester product with an enantiomeric excess of C₁-C₄ alkyl (R)-3-aminobutanoate. The (R)-selective ω-transaminase may be selected from the group consisting of ATA enzymes Prozomix ATA 234, Prozomix ATA 241, Prozomix ATA 254 or Prozomix ATA 261 supplied by Prozomix Limited or an (R)-selective wild type enzyme isolated from Neosartorya fischeri, Aspergillus fumigatus or Aspergillus terreus.

The method may include a further step of reducing the C₁-C₄ alkyl (R)-3-aminobutanoate product to (R)-3-aminobutyric acid by base-catalysed hydrolysis with a strong base in an alcohol solvent, such as KOH in methanol. Alternatively, the C₁-C₄ alkyl (R)-3-aminobutanoate may be reduced to (R)-3-aminobutan-1-ol, which is useful for the synthesis of dolutegravir. As before, the reduction of the ester to an alcohol can be done with excess sodium borohydride in tetrahydrofuran with additives lithium chloride or calcium chloride and at a temperature ranging between room temperature and 70° C.

To produce the corresponding (S)-enantiomers instead of the (R)-enantiomers, an (S)-selective ω-transaminase may be used to produce an amino ester product with an enantiomeric excess of C₁-C₄ alkyl (S)-3-aminobutanoate. The C₁-C₄ alkyl (S)-3-aminobutanoate may be reduced to either (S)-3-aminobutyric acid or to (S)-3-aminobutan-1-ol in the same manner as described with reference to the (R)-enantiomers.

Dolutegravir may be prepared in a method comprising contacting C₁-C₄ alkyl 3-oxobutyrate with an (R)-selective ω-transaminase in the presence of an amino donor to produce substantially enantiomerically pure C₁-C₄ alkyl (R)-3-aminobutanoate. Then reducing the C₁-C₄ alkyl (R)-3-aminobutanoate to (R)-3-aminobutan-1-ol and finally converting the (R)-3-aminobutan-1-ol to dolutegravir (i.e., using (R)-3-aminobutan-1-ol to prepare dolutegravir). It is preferred that the substrate is ethyl or isopropyl 3-oxobutyrate and that the (R)-selective ω-transaminase is an enzyme selected from the ATA enzymes encoded Prozomix ATA 234, Prozomix ATA 241, Prozomix ATA 254 or Prozomix ATA 261 supplied by Prozomix Limited, more preferably Prozomix ATA 241.

An exemplary method of preparing the key (R)-amino alcohol intermediate (6) for dolutegravir, from an achiral ketone, isopropyl acetoacetate (7), with (R)-selective ω-transaminase enzymes is demonstrated in Scheme 5. Synthetic routes to other useful (R) or (S) β-homoalaninol and β-homoalanine intermediates are also demonstrated in Scheme 5.

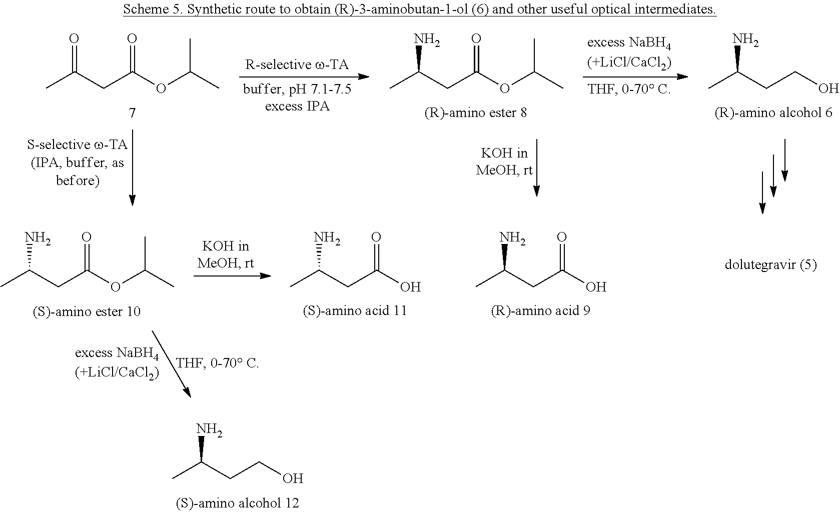

The β-keto ester (7) starting material can be produced cost-effectively by the transesterification of methyl-or ethyl acetoacetate with isopropanol, or directly from diketene and isopropanol, by known methods. Advantageously, the methods of reducing the (R) or (S) amino-ester intermediates to the corresponding amino alcohol or amino acid intermediates do not result in significant racemization.

### EXAMPLES

### Methods

### 1. Transamination Reaction

A reaction volume of 1 ml is prepared in a 2 ml eppendorf tube. A buffer is prepared consisting of 100 mM potassium dihydrogen phosphate buffer with the pH adjusted to 7.5 with concentrated NaOH. The buffer further includes 0.5 g/L of pyridoxal phosphate (PLP) as co-factor. Isopropyl amine is added to the buffer to a final concentration of 1 M and the pH is adjusted to 7.1-7.5, most preferably 7.5. Approximately 5 mg of enzyme is added to the buffer. Finally, substrate (ester of formula I) is added to produce a final substrate concentration of 20 mM. The eppendorf tubes are then incubated at 30° C. for 30 min to 24 h. Brine, sodium bicarbonate and acetonitrile are added to stop the reaction after incubation for the specified time. The top (organic) layer containing the product is separated via centrifugation and aspiration.

### 2. Product Analysis

After extraction of the product, the acetonitrile is evaporated in vacuo to dry the product. The product is dissolved in a minimum amount of DCM, to which excess triethylamine is added, followed by excess p-nitrobenzoyl chloride. After reaction at room temperature overnight, the mixture is evaporated in vacuo and purified by semi-preparative TLC. The product band is scraped into a vial and dissolved in 5% MeOH/95% CH₃CN. After filtering through a cotton wool plug, the derivatised amide product is analysed directly by UPLC/HRMS (or UV detection at 254 nm).

### Results

### 1. Biocatalytic Conversion of Isopropyl 2-Oxobutyrate, to Specific Enantiomers of Isopropyl 2-Aminobutyrate

ATA enzyme screening was carried out with the substrate, isopropyl 2-oxobutyrate. Achiral HPLC analysis was used to determine the percentage conversion of the substrate to the desired isopropyl 2-aminobutyrate product.

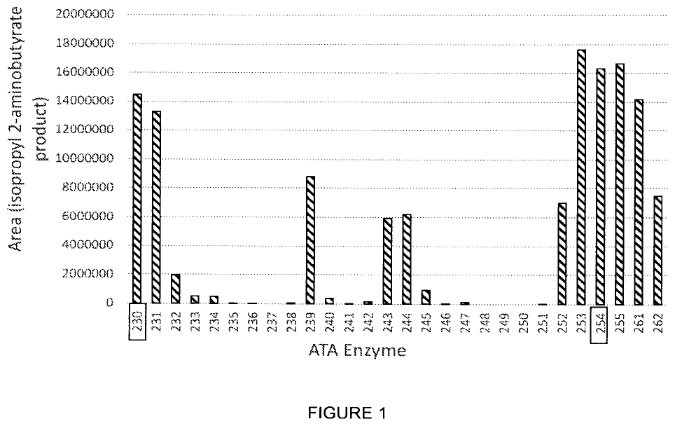

FIG. 1 is a plot showing the amount of isopropyl 2-aminobutyrate product obtained with different Prozomix ATA enzymes.

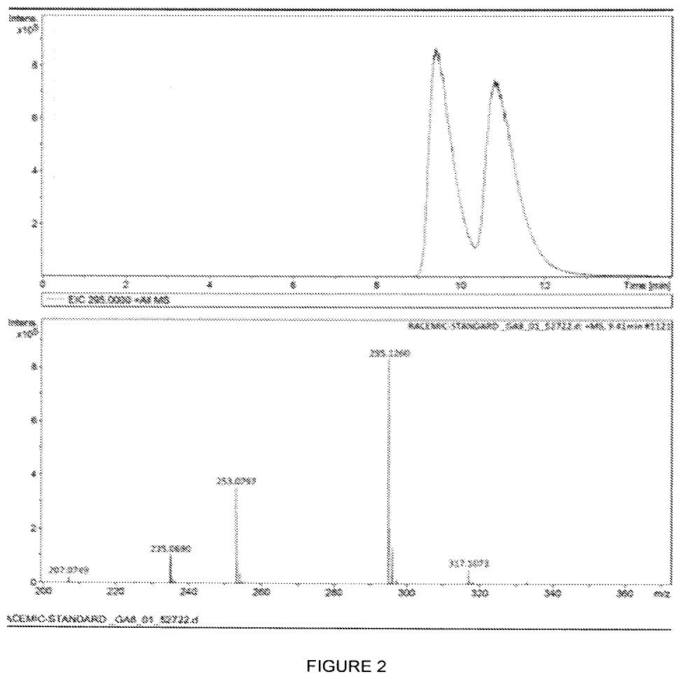

FIG. 2 is an Ultra-high Performance Liquid Chromatography-MS (UPLC-MS) analysis of derivatised (chemically synthesised) racemic amine (isopropyl 2-aminobutyrate) product. Table 1 includes the results of the analysis.

UPLC Method: Column=Astec Chirobiotic T (10 cm×4.6 mm); isocratic elution using 95% water (+0.1% formic acid) and 5% acetonitrile (+0.1% formic acid); flow rate=1.0 ml/min.

TABLE 1
UPLC-MS analysis of racemic isopropyl 2-aminobutyrate

| | | | | | % | % | |
|---|---|---|---|---|---|---|---|
| | Calculated | Found | Molecular | Retention | Area | Area | |
| Compound | mass | mass | ion | time | S | R | % ee |
| | 294.1216 | 295.1260 | [M + H]⁺ | S = 9.40 | 50 | 0 | 0% |
| | C₁₄H₁₈N₂O₅ | | | R = 10.74 | | | (racemic) |

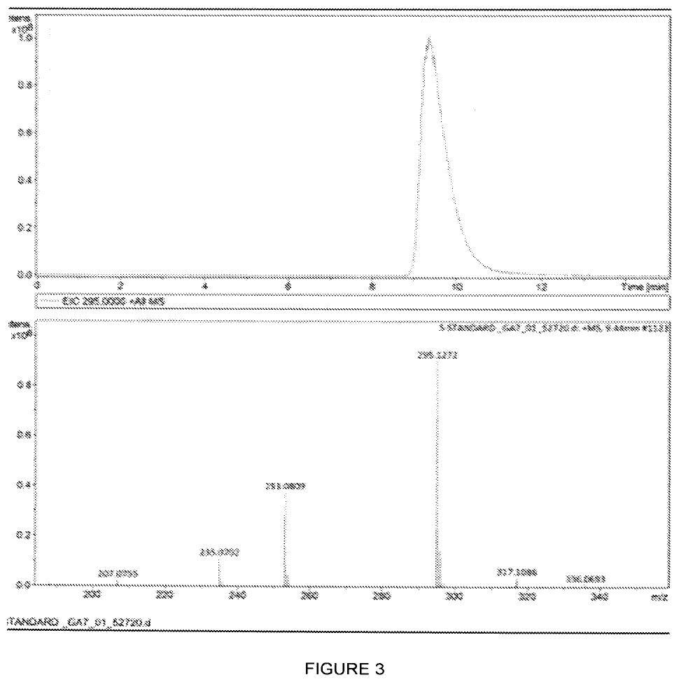

FIG. 3 is the UPLC-MS analysis of the derivatised (chemically synthesised) isopropyl(S)-2-aminobutyrate. The results of the analysis are in Table 2.

TABLE 2
UPLC-MS analysis of (S)-isopropyl 2-aminobutyrate

| | Calculated | Found | Molecular | Retention | % Area | % Area | |
|---|---|---|---|---|---|---|---|
| Compound | mass | mass | ion | time | S | R | % ee |
| | 294.1216 | 295.1272 | [M + H]⁺ | S = 9.40 | >99.9 | <0.1 | >99% |
| | C₁₄H₁₈N₂O₅ | | | | | | S |

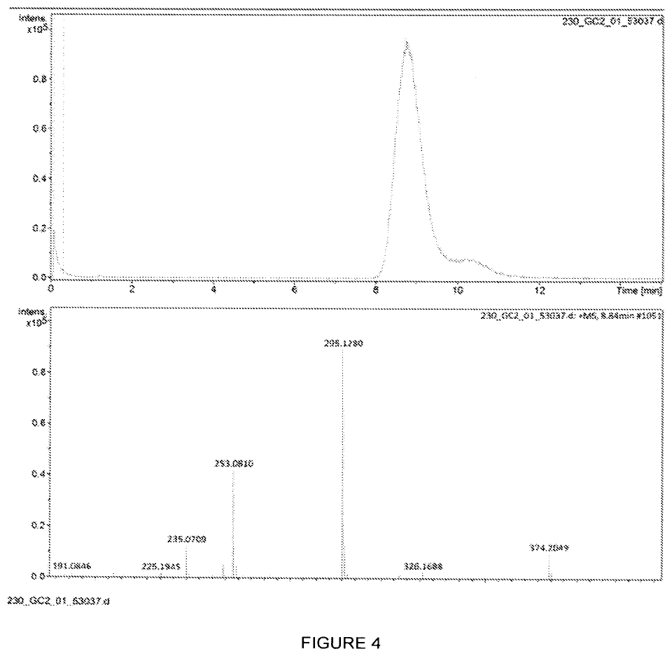

FIG. 4 is the UPLC-MS analysis of derivatised product from Prozomix ATA 230. The results of this analysis are in Table 3.

TABLE 3
UPLC-MS analysis of derivatised product from Prozomix ATA 230

| | Calculated | Found | Molecular | Retention | % Area | % Area | |
|---|---|---|---|---|---|---|---|
| Enzyme | mass | mass | ion | time | S | R | % ee |
| 230 | 294.1216 | 295.1280 | [M + H]⁺ | S = 8.73 | 99.50 | 0.50 | 99% |
| | C₁₄H₁₈N₂O₅ | | | R = 10.35 | | | S |

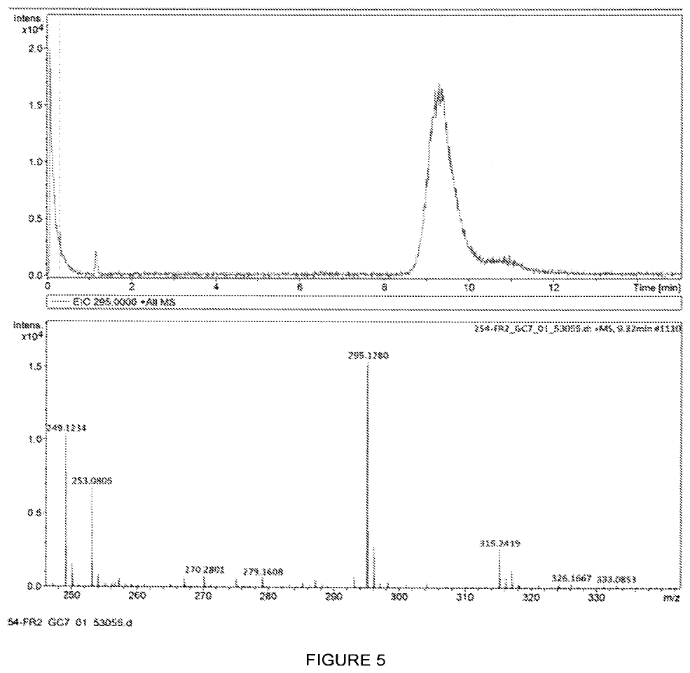

FIG. 5 is the UPLC-MS analysis of derivatised product from Prozomix ATA 254. The results of this analysis are in Table 4.

TABLE 4
UPLC-MS analysis of derivatised product from Prozomix ATA 254

| | | | | | % | % | |
|---|---|---|---|---|---|---|---|
| | Calculated | Found | Molecular | Retention | Area | Area | |
| Enzyme | mass | mass | ion | time | S | R | % ee |
| 254 | 294.1216 | 295.1280 | [M + H]⁺ | S = 9.57 | 99.50 | 0.50 | Approx. |
| | C₁₄H₁₈N₂O₅ | | | R = 11.02 | | | 99% S |

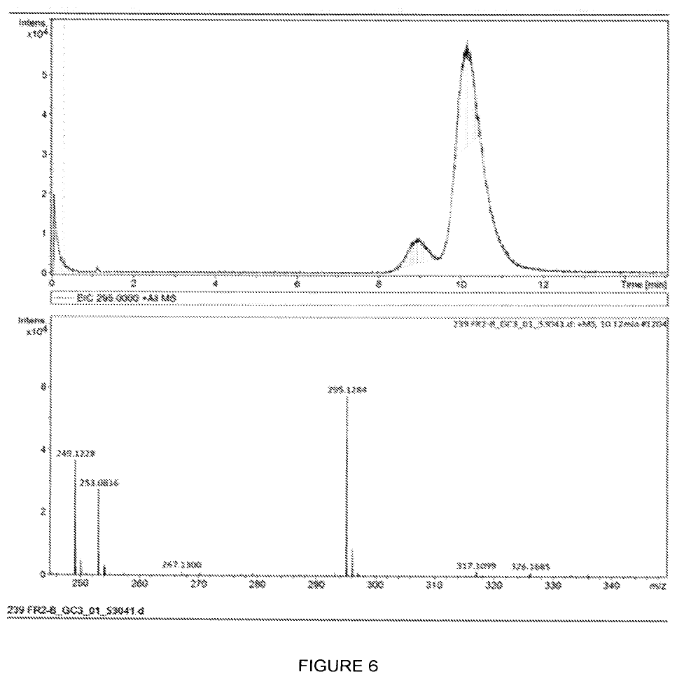

FIG. 6 is the UPLC-MS analysis of derivatised product from Prozomix ATA 239. The results of this analysis are in Table 5.

TABLE 5
UPLC-MS analysis of derivatised product from Prozomix ATA 239

| | Calculated | Found | Molecular | Retention | % Area | % Area | |
|---|---|---|---|---|---|---|---|
| Enzyme | mass | mass | ion | time | S | R | % ee |
| 239 | 294.1216 | 295.1284 | [M + H]⁺ | S = 8.99 | 14.85 | 85.15 | 70.20% |
| | C₁₄H₁₈N₂O₅ | | | R = 10.14 | | | R |

### 2. Biocatalytic Conversion of Isopropyl 3-Oxobutyrate to Isopropyl 3-Aminobutyrate

The ATA enzyme-catalysed reaction was carried out on the substrate, isopropyl 3-oxobutyrate, using isopropyl amine as the amino donor and with the reaction conditions specified in the methods above and shown in Scheme 6.

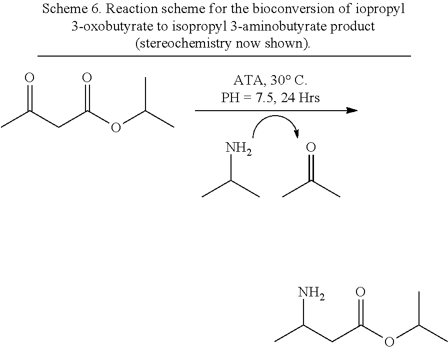

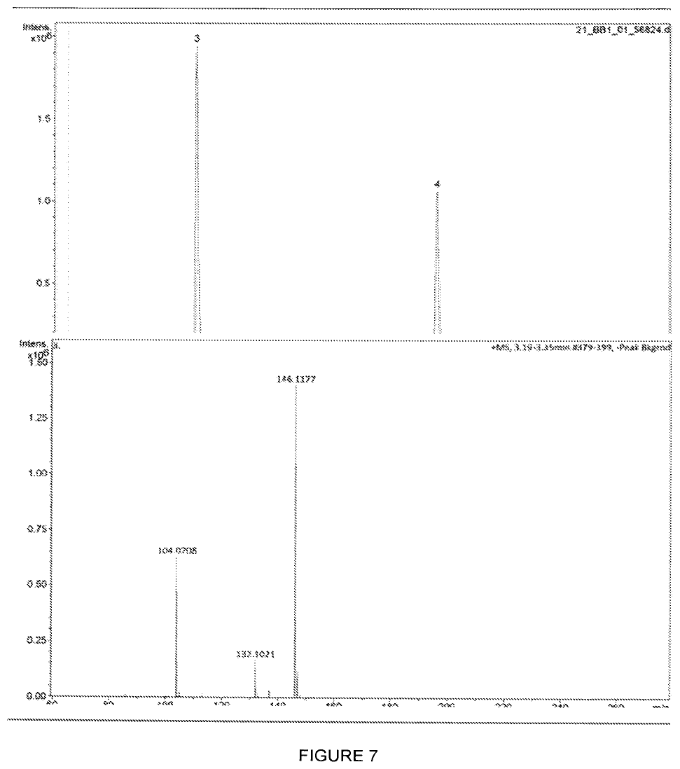

FIG. 7 is the LC-MS analysis of the derivatised isopropyl 3-aminobutyrate product from Prozomix ATA 235. The product elutes as two distinct peaks representing the protonated and unprotonated product.

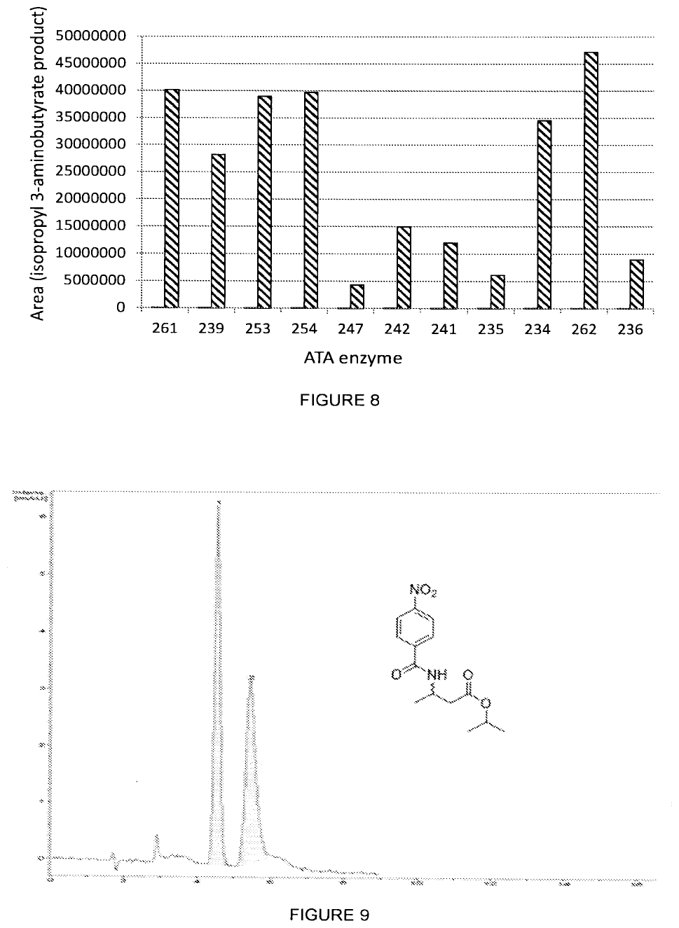

Enzyme screening of twenty-eight enzymes revealed eleven enzymes that are active against the substrate. FIG. 8 is a plot comparing the area (percentage conversion) of the amine product peaks from the LC-MS of the enzyme-catalysed reaction with the eleven active ATA enzymes.

The R-enantiomer, isopropyl (R)-3-aminobutyrate can be obtained in high enantiomeric excess with wild type enzymes which are known to always produce the R-enantiomer.

FIG. 9 is an Ultra-high Performance Liquid Chromatography-UV (254 nm) (UPLC-UV detection) analysis of derivatised (chemically synthesised) racemic amine (isopropyl 3-aminobutyrate) product. Table 6 includes the results of the analysis which is an approximately 50% (R) and 50% (S) mixture.

UPLC Method: Column=Chiralpak AD-RH (15 cm×4.6 mm); isocratic elution using 50% water (+0.1% formic acid) and 50% acetonitrile (+0.1% formic acid); flow rate=1.0 ml/min.

TABLE 6
UPLC-UV(254 nm) analysis of derivatised,
racemic isopropyl 3-aminobutyrate

| Name | RT [min] | Area |
|---|---|---|
| RAC- | 4.55 | 69.515 |
| STANDARD | 5.46 | 64.173 |

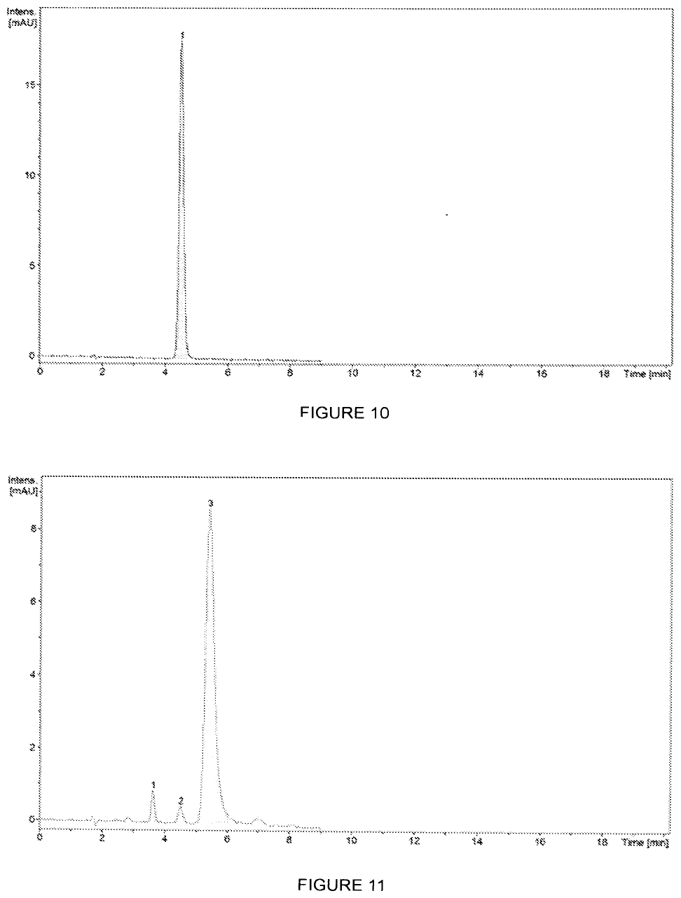

FIG. 10 is the UPLC-UV (254 nm) analysis of the derivatised (chemically synthesised) isopropyl (S)-3-aminobutyrate. The results of the analysis are in Table 7 which indicate that >99% enantiomeric excess of the (S)-product was obtained. The (R)-product must elute at RT=5.46 as per Table 6, but was not detected.

TABLE 7
UPLC-UV(254 nm) analysis of derivatised
(S)-isopropyl 2-aminobutyrate.

| Name | RT [min] | Area |
|---|---|---|
| S-STANDARD | 4.53 | 184.22 (a single peak found) |

FIG. 11 is the UPLC-UV (254 nm) analysis of derivatised product from Prozomix ATA 254. The results of this analysis are in Tables 8 and 9.

TABLE 8
UPLC-UV(254 nm) analysis of derivatised
product from Prozomix ATA 254

| Enzyme | RT [min] | Area | Int. Type | I | |
|---|---|---|---|---|---|
| 254 | 3.64 | 6.6124 | Chromatogram | 1 | 5199.7 |
| | (derivatisation | | | | |
| | impurity, p- | | | | |
| | nitrobenzoic acid) | | | | |
| | 4.53 | 5.1066 | Chromatogram | 0 | 2709.9 |
| | 5.42 | 180.2397 | Chromatogram | 9 | 51150.5 |

TABLE 9
Enantiomeric excess calculation with Prozomix ATA 254

| % S area | % R area | % ee |
|---|---|---|
| 2.76 | 97.24 | 94.48% R |

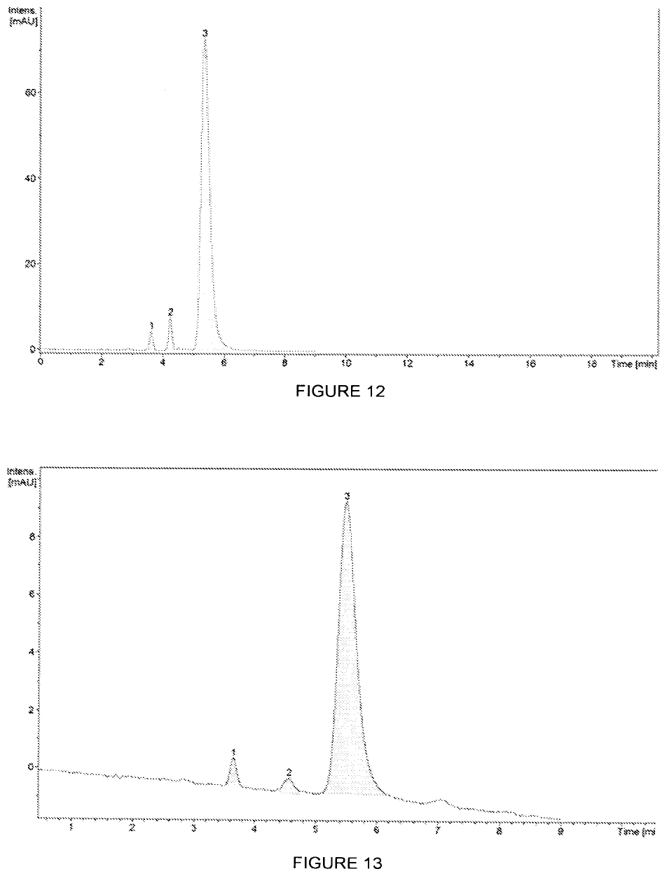

FIG. 12 is the UPLC-UV (254 nm) analysis of derivatised product from Prozomix ATA 261. The results of this analysis are in Table 10 and 11.

TABLE 10
UPLC-UV(254 nm) analysis of derivatised
product from Prozomix ATA 261

| Enzyme | RT [min] | Area |
|---|---|---|
| 261 | 4.26 | 56.095 |
| | 5.37 | 1484.484 |

TABLE 11
Enantiomeric excess calculation with Prozomix ATA 261.

| % S area | % R area | % ee |
|---|---|---|
| 3.64 | 96.36 | 92.72% R |

FIG. 13 is the UPLC-UV (254 nm) analysis of derivatised product from Prozomix ATA 234. The results of this analysis are in Tables 12 and 13.

TABLE 12
UPLC-UV(254 nm) analysis of derivatised
product from Prozomix ATA 234

| Enzyme | RT [min] | Area |
|---|---|---|
| 234 | 3.65(derivatisation | 7.2418 |
| | impurity, p-nitrobenzoic | |
| | acid) | |
| | 4.57 | 7.3283 |
| | 5.49 | 213.9455 |

TABLE 13
Enantiomeric excess calculation with Prozomix ATA 234

| % S area | % R area | % ee |
|---|---|---|
| 3.31% | 96.69% | 93.39% R |

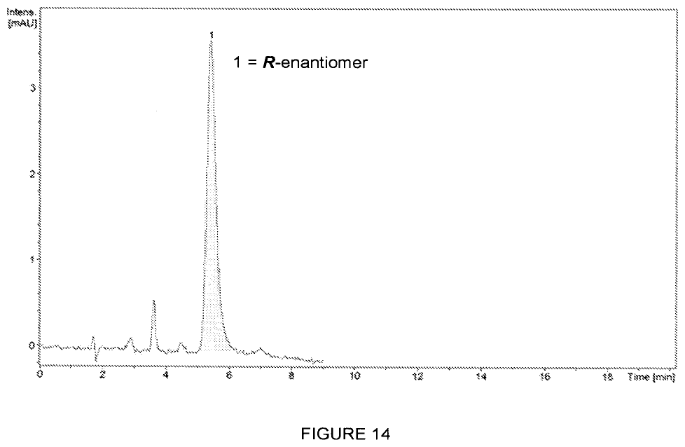

FIG. 14 is the UPLC-UV (254 nm) analysis of derivatised product from Prozomix ATA 241. The results of this analysis are in Table 14.

TABLE 14
UPLC-UV(254 nm) analysis of derivatised
product from Prozomix ATA 241.

| Enzyme | RT [min] | Area | Int. Type | I | |
|---|---|---|---|---|---|
| 241 | 5.42 | 74.981 | Chromatogram | 4 | 25898.1 |

A single peak eluted at retention time of 5.42 minutes therefore >99% R enantiomeric excess (ee) was achieved with Prozomix ATA 241.

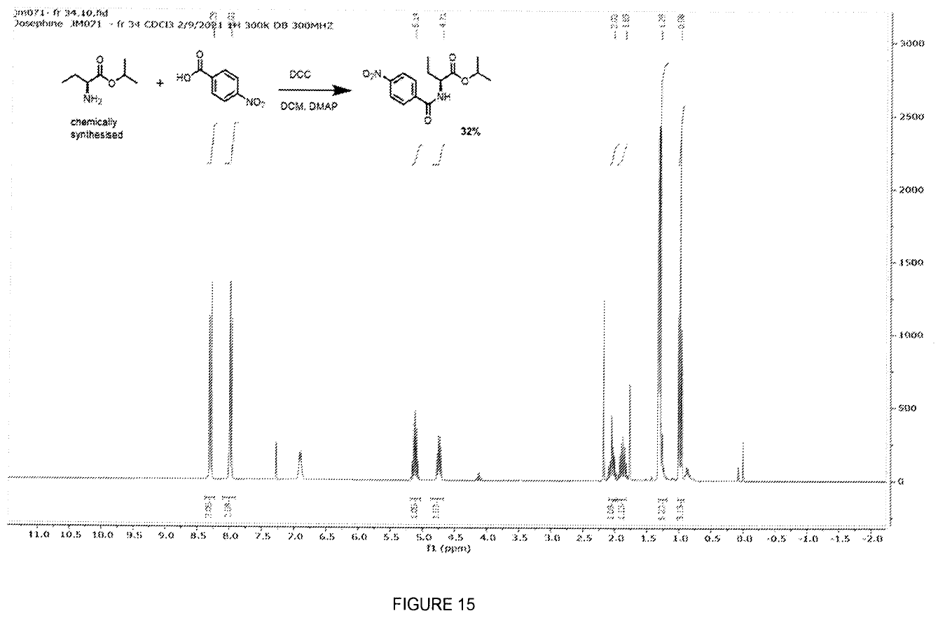

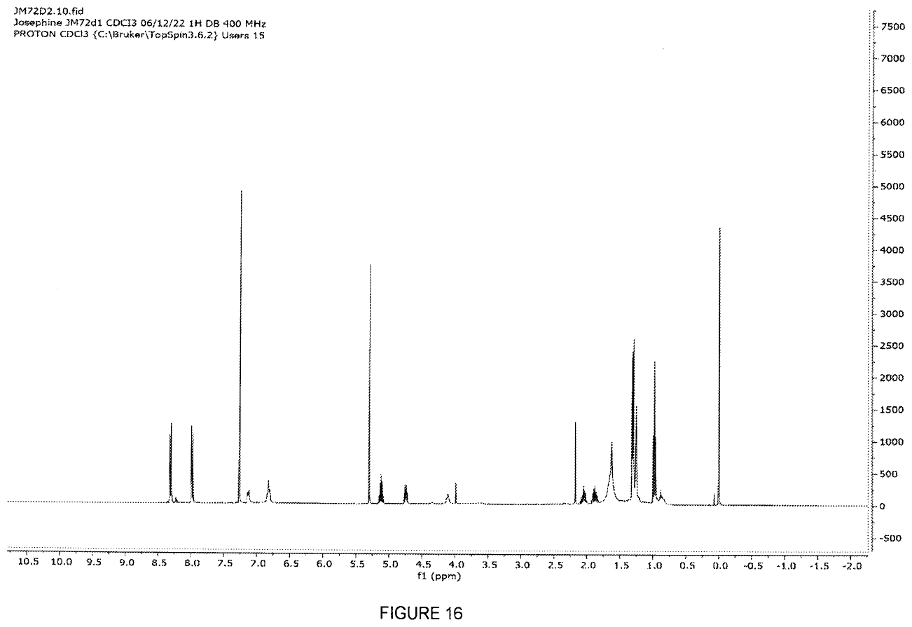

FIG. 15 is the proton nuclear magnetic resonance (NMR) spectrum of chemically synthesised 4-nitrobenzamide derivative of the (S)-2-amino isopropyl ester. FIG. 16 is a proton NMR spectrum of the 4-nitrobenzamide derivative of the Prozomix ATA 254 enzyme produced (S)-2-amino isopropyl ester, for comparison to FIG. 15.

The methods described herein provide cost-efficient and “greener” synthetic routes to non-proteinogenic amino acids such as (S)-homoalanine as a precursor for preparing levetiracetam and brivaracetam and specific (S) or (R) amino alcohol enantiomers which serve as precursors for preparing ethambutol or dolutegravir, for example. Advantageously, the starting materials used, such as ethyl or isopropyl 2-oxobutyrate (for ethambutol, levetiracetam, brivaracetam) and ethyl or isopropyl acetoacetate (for dolutegravir), are more cost-effective to produce or obtain in comparison to the starting materials currently used to produce these drugs in industry.

In general, the methods for preparing chiral amine pharmaceutical intermediates avoid starting from, or producing, free amino acid intermediates, as in current approaches. Free amino acids are not as readily purified and processed in conventional synthesis procedures as amino esters are, due to the need for purification via costly and laborious ion exchange chromatography, followed by energy-consuming evaporation procedures. The amino ester enantiomers produced in the biocatalytic stereoselective methods described herein can be recovered more easily and in higher yields.

The foregoing description has been presented for the purpose of illustration; it is not intended to be exhaustive or to limit the invention to the precise forms disclosed. Persons skilled in the relevant art can appreciate that many modifications and variations are possible in light of the above disclosure. For example, it will be apparent to those skilled in the art that the substrate for the enantioselective ω-transaminase catalysed step may include different R groups having a similar size and surface polarity to the currently specified R groups, provided that the activity (percentage conversion) of the enzyme is not substantially reduced. It will also be understood by those skilled in the art that the type of enantioselective ω-transaminase used, the reaction conditions and the types and concentration of the reagents in the contact step and its duration may be varied to obtain a product with the maximum enantiomeric purity (substantially pure enantiomer). It will also be apparent to persons skilled in the art that the enantiomeric selectivity of a specific ω-transaminase (i.e., whether it is (S)- or (R)-selective) may depend on the type of keto ester substrate.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention set forth in any accompanying claims.

Finally, throughout the specification and any accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

1. A method for preparing chiral amines, comprising contacting an ester of formula I: 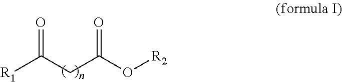
in which R₁ is a methyl or an ethyl group;
R₂ is a linear or branched C₁-C₄ alkyl group; and
n is 0 or 1
with an enantioselective ω-transaminase in the presence of an amino donor so that the ω-transaminase catalyses the enantioselective transfer of an amino group from the amino donor to an α-ketone group when n is 0 or an β-ketone group when n is 1 of the ester of formula I to produce an amino ester product with enantiomeric excess of a selected enantiomer.

2. The method as claimed in claim 1, wherein the enantiomeric excess of the selected enantiomer is at least 95%.

3. The method as claimed in claim 1, wherein the enantioselective ω-transaminase is an (S)-selective ω-transaminase for a selected ester of formula I to produce enantiomeric excess of an (S)-amino ester enantiomer.

4. The method as claimed in claim 1, wherein the enantioselective ω-transaminase is an (R)-selective ω-transaminase for a selected ester of formula I to produce enantiomeric excess of an (R)-amino ester enantiomer.

5. The method as claimed in claim 1, wherein the amino donor is isopropyl amine.

6. The method as claimed in claim 1, wherein the contacting occurs in a buffer solution including pyridoxal phosphate (PLP) as co-factor and having a pH ranging between 7.1 and 7.5 and at a temperature within the range of 20° C. to 40° C.

7. The method as claimed in claim 1, which further includes reducing the selected amino ester enantiomer to the corresponding amino acid or amino alcohol in a manner which substantially avoids racemization occurring.

8. The method as claimed in claim 1, wherein R₂ is a methyl, ethyl, isopropyl, n-butyl, sec-butyl, isobutyl, or tert-butyl group.

9. The method as claimed in claim 1, wherein when n is 0 and R₁ is an ethyl group so that the ester of formula I is a C₁-C₄ alkyl 2-oxobutyrate, and wherein the ω-transaminase is an (S)-selective ω-transaminase configured to catalyse the production of an amino ester product with an enantiomeric excess of C₁-C₄ alkyl (S)-2-aminobutanoate.

10. The method as claimed in claim 9, wherein R₂ is an ethyl or an isopropyl group and the (S)-selective @-transaminase for ethyl or isopropyl 2-oxobutyrate is the (S)-selective amine transaminases (ATA) enzymes Prozomix ATA 230 or Prozomix ATA 254 to obtain ethyl or isopropyl (S)-2-aminobutanoate in an enantiomeric excess of at least 99%.

11. The method as claimed in claim 10, which further includes hydrolysing the ethyl or isopropyl (S)-2-aminobutanoate to (S)-2-aminobutyric acid.

12. The method as claimed in claim 10, which further includes reducing the ethyl or isopropyl (S)-2-aminobutanoate to (S)-2-aminobutan-1-ol.

13. The method as claimed in claim 1, wherein n is 0 and R₁ is an ethyl group, and wherein the ω-transaminase is an (R)-selective ω-transaminase configured to catalyse the production of an amino ester product with enantiomeric excess of C₁-C₄ alkyl (R)-2-aminobutanoate.

14. The method as claimed in claim 13, wherein R₂ is an ethyl or an isopropyl group and the (R)-selective ω-transaminase for ethyl or isopropyl 2-oxobutyrate is an (R)-selective ATA enzyme Prozomix ATA 239 or a wild-type (R)-selective transaminase isolated from one or more of Neosartorya fischeri, Aspergillus fumigatus or Aspergillus terreus to obtain the ethyl or isopropyl (R)-2-aminobutanoate in an enantiomeric excess of at least 70%.

15. The method as claimed in claim 14, which further includes hydrolysing the ethyl or isopropyl (R)-2-aminobutanoate to (R)-2-aminobutyric acid.

16. The method as claimed in claim 14, which further includes reducing the ethyl or isopropyl (R)-2-aminobutanoate to (R)-2-aminobutan-1-ol.

17. The method as claimed in claim 1, which further includes preparing the ester of formula I in which n is 0 and R₁ is an ethyl group by reacting diethyl or diisopropyl oxalate with ethyl magnesium bromide to produce an ethyl or isopropyl 2-oxobutyrate.

18. The method as claimed in claim 1, wherein n is 1 and R₁ is a methyl group so that the ester of formula I is an C₁-C₄ alkyl 3-oxobutyrate, and wherein the ω-transaminase is an (R)-selective ω-transaminase configured to catalyse the production of an amino ester product with enantiomeric excess of C₁-C₄ alkyl ((R)-3-aminobutanoate.

19. The method as claimed in claim 18, wherein R₂ is an ethyl or isopropyl group and the (R)-selective ω-transaminase for ethyl or isopropyl 3-oxobutyrate is an (R)-selective ATA enzyme selected from one or more of Prozomix ATA 234, Prozomix ATA 241, Prozomix ATA 254 or Prozomix ATA 261 or an (R)-selective wild type enzyme isolated from Neosartorya fischeri, Aspergillus fumigatus or Aspergillus terreus.

20. The method as claimed in claimed in claim 19, which further includes hydrolysing the ethyl or isopropyl (R)-3-aminobutanoate to (R)-3-aminobutyric acid.

21. The method as claimed in claim 19, which further includes reducing the ethyl or isopropyl (R)-3-aminobutanoate to (R)-3-aminobutan-1-ol.

22. The method as claimed in claim 1, wherein n is 1, R₁ is a methyl group and the ω-transaminase is an (S)-selective ω-transaminase configured to catalyse the production of an amino ester product with enantiomeric excess of C₁-C₄ alkyl (S)-3-aminobutanoate.

23. The method as claimed in claim 22, which further includes hydrolysing the C₁-C₄ alkyl (S)-3-aminobutanoate to (S)-3-aminobutyric acid.

24. The method as claimed in claim 22, which further includes reducing the C₁-C₄ alkyl (S)-3-aminobutanoate to (S)-3-aminobutan-1-ol.
